(19) United States
(12) Patent Application Publication
(10) Pub. No.: US 2006/0041314 A1
(43) Pub. Date: Feb. 23, 2006

(54) Artificial disc prosthesis

(76) Inventor: Thierry Millard, Cestas (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/208,663

(22) Filed: Aug. 22, 2005

Related U.S. Application Data
(60) Provisional application No. 60/603,060, filed on Aug. 20, 2004.

(51) Int. Cl. A61F 2/44

(52) U.S. Cl. 623/17.16; 623/17.15

(57) ABSTRACT

An intervertebral disc having a first end plate, a second end plate and a core. The first end plate including a top surface and a bottom spherical surface. The second end plate having a lower surface and an upper surface, the upper surface including at least one protrusion having a length and a width extending upward from said upper surface. The core having a concave surface and a second surface. The core adapted for being disposed between the first end plate and the second end plate.

### CROSS REFERENCE TO RELATED APPLICATIONS

This application claims the benefit of the filing date of U.S. Provisional Patent Application No. 60/603,060 filed Aug. 20, 2004, the disclosure of which is hereby incorporated herein by reference.

### BACKGROUND OF THE INVENTION

This invention relates generally to a spinal implant assembly for implantation into the intervertebral space between adjacent vertebral bones to simultaneously provide stabilization and continued flexibility and proper anatomical motion, and more specifically to such a device that has the ability to provide sliding action between elements of the device.

The bones and connective tissue of an adult human spinal column consists of more than twenty discrete bones coupled sequentially to one another by a tri-joint complex that consists of a disc and the two posterior facet joints. The discs of adjacent bones are cushioned by spacers referred to as intervertebral discs. These more than twenty bones are anatomically categorized as being members of one of four classifications: cervical, thoracic, lumbar, or sacral. The cervical portion of the spine, which comprises the top of the spine, up to the base of the skull, includes the first seven vertebrae. The intermediate twelve bones are the thoracic vertebrae, and connect to the lower spine comprising the five lumbar vertebrae. The base of the spine is the sacral bones (including the coccyx). The component bones of the cervical spine are generally smaller than those of the thoracic spine, which are in turn smaller than those of the lumbar region. The sacral region connects laterally to the pelvis.

The spinal column is highly complex in that it includes these more than twenty bones coupled to one another, housing and protecting critical elements of the nervous system having innumerable peripheral nerves and circulatory bodies in close proximity. In spite of these complications, the spine is a highly flexible structure, capable of a high degree of curvature and twist in nearly every direction.

Genetic or developmental irregularities, trauma, chronic stress, tumors, and degenerative wear are a few of the causes that can result in spinal pathologies for which surgical intervention may be necessary. A variety of systems have been disclosed in the art that achieve immobilization and/or fusion of adjacent bones by implanting artificial assemblies in or on the spinal column. The region of the back that needs to be immobilized, as well as the individual variations in anatomy, determine the appropriate surgical protocol and implantation assembly. With respect to the failure of the intervertebral disc, the interbody fusion cage has generated substantial interest because it can be implanted laparoscopically into the anterior of the spine, thus reducing operating room time and patient recovery time scarification.

Many intervertebral body cages comprise tubular metal body having an external surface threading. They are inserted transverse to the axis of the spine, into preformed cylindrical holes at the junction of adjacent vertebral bodies. Two cages may be inserted side by side with the external threads threading into the upper and lower surfaces of the adjacent vertebral bones. The cages may include holes through which the adjacent bones are to grow. Additional materials, for example autogenous bone graft materials, may be inserted into the hollow interior of the cage to incite or accelerate the growth of the bone into the cage. End caps are often utilized to hold the bone graft material within the cage.

These cages of the prior art have enjoyed medical success in promoting fusion and approximating proper disc height. It is, however, important to note that the fusion of the adjacent bones is an incomplete solution to the underlying pathology as it does not cure the ailment, but rather simply masks the pathology under a stabilizing bridge of bone. Thus, bone fusion limits the overall flexibility of the spinal column and artificially constrains the normal motion of the patient. This constraint can cause collateral injury to the patient's spine as additional stresses of motion, normally borne by the now-fused joint, are transferred onto the nearby facet joints and intervertebral discs. It would therefore, be a considerable advance in the art to provide an implant assembly which does not promote fusion, but rather, which mimics the biomechanical action of the natural disc cartilage, thereby permitting continued normal motion and stress distribution.

Some artificial intervertebral discs have been designed that permit greater flexibility of the spine, specifically of adjacent vertebral bodies. See, for example, that which is detailed in U.S. patent application Ser. No. 10/256,160 (filed Sep. 26, 2002) entitled “Artificial Intervertebral Disc,” which is a continuation-in-part application of U.S. patent application Ser. No. 10/175,417 (filed Jun. 19, 2002) entitled “Artificial Intervertebral Disc Utilizing a Ball Joint Coupling”, which is a continuation-in-part application of U.S. patent application Ser. No. 10/151,280 (filed May 20, 2002) entitled “Tension Bearing Artificial Disc Providing a Centroid of Motion Centrally Located Within an Intervertebral Space”, which is a continuation-in-part application of both U.S. patent application Ser. No. 09/970,479 (filed Oct. 4, 2001) entitled “Intervertebral Spacer Device Utilizing a Spirally Slotted Belleville Washer Having Radially Extending Grooves” as well as U.S. patent application Ser. No. 10/140,153, (filed May 7, 2002) entitled “Artificial Intervertebral Disc Having a Flexible Wire Mesh Vertebral Body Contact Element”, the former being a continuation-in-part application of U.S. patent application Ser. No. 09/968,046 (filed Oct. 1, 2001) entitled “Intervertebral Spacer Device Utilizing a Belleville Washer Having Radially Extending Grooves” and the latter being a continuation-in-part application of both U.S. patent application Ser. No. 09/970,479 (detailed above) as well as U.S. patent application Ser. No. 10/128,619 (filed Apr. 23, 2002) entitled “Intervertebral Spacer Having a Flexible Wire Mesh Vertebral Body Contact Element”, which is a continuation-in-part application of both U.S. patent application Ser. No. 09/906,119 (filed Jul. 16, 2001) and entitled “Trial Intervertebral Distraction Spacers” as well as U.S. patent application Ser. No. 09/982,148 (filed Oct. 18, 2001) and entitled “Intervertebral Spacer Device Having Arch Shaped Spring Elements,” the disclosures of which are incorporated herein by reference as if fully set forth herein. But still, what is needed is artificial discs that closely mimic the natural movement of the spine.

The present invention relates generally to artificial disc replacements.

### BRIEF DESCRIPTION OF THE DRAWINGS

FIG. 1 is a cross-sectional front view of an anterior side of a first embodiment of a disc according to the present invention;

FIG. 2 illustrates a force being applied to the disc of FIG. 1;

FIG. 3 is a cross-sectional front view of a medial side of the disc of FIG. 1;

FIG. 4 illustrates a force being applied to the disc of FIG. 3;

FIG. 5 is a cross-sectional front view of an anterior side of a second embodiment of a disc according to the present invention;

FIG. 6 illustrates a force being applied to the disc of FIG. 1;

FIG. 7 is a cross-sectional view of an anterior side of the disc of FIG. 5;

FIG. 8 illustrates a force being applied to the disc of FIG. 7;

FIG. 9 is a cross-sectional perspective view of the embodiment shown in FIG. 1; and

FIG. 10 is a cross-sectional perspective view of the embodiment shown in FIG. 5.

### DETAILED DESCRIPTION

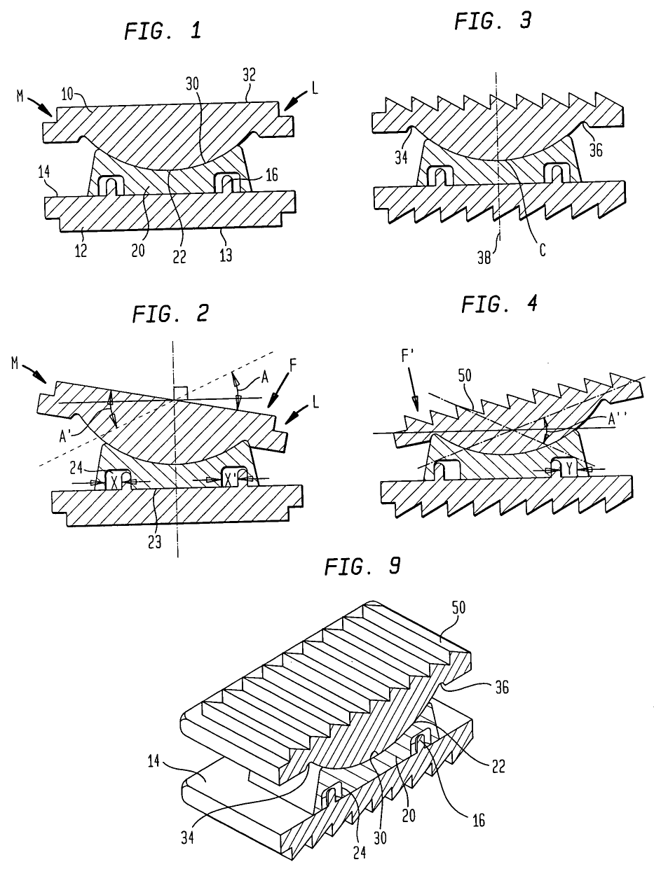

As shown in FIGS. 1-4, the present invention is directed to an intervertebral disc arthroplasty device 1 having two end plate members referenced generally at 10 and 12 in FIG. 1. FIGS. 1 and 2 are cross-sectional front views of the anterior side of one embodiment of the present invention. The disc is designed to be disposed between two adjacent vertebrae and includes a medial side M and a lateral side L. The end plate members are preferably comprised of a CoCrMo alloy. The end plate members may also be comprised of most other biologically compatible materials. And it may be coated with or made such that surfaces thereof include bone growth inducing structures, compositions or substances including but not limited to bone morphogenetic proteins, hydroxyapetite, calcium phosphates, polymers with bone inducing materials or compositions therein or thereunder, etc. . . . Antimicrobial and anesthetic agents, immunosuppressive agents and various other pain killing and bioactive materials.

The bottom end plate member 12 may have a substantially flat top surface 14 with a plurality of protrusions 16 extending outwardly above top surface 14. Although reference words such as “upward” and “downward” may be used to describe the relationship between two features, the use of the terms is in no way meant to describe a gravitational reference point.

A core 20 may be disposed on top surface 14 of end plate 12. In a preferred embodiment, core 20 is comprised of a compressionable material. For example, the core 20 is preferably made from an ultra-high molecular weight polyethylene. The core may also be comprised of other materials such as ceramic and other plastics and even metal.

The core preferably includes a concave arch 22 remote from end plate 12. The concave arch 22 forms a cup. Core 20 also preferably includes an array of indents 24. Indents 24 are disposed on bottom surface 23 of core 20. The array of indents 24 is designed to receive protrusions 16 emanating upward from end plate 12. The array of indents 24 have a larger opening as compared to the length and width of the protrusions 16. This design enables core 20 to slide along end plate 12, as will later be described. The concave arch besides being spherical may also be in the shape of an elongated cylinder.

Top end plate 10 preferably includes a spherical convex lower surface 30 and an upper surface 32. Convex lower surface 30 has a concavity substantially equal to the concavity of concave surface 22 of core 20 such that a radius of the convex lower surface 30 can reside in the cup of the core. In the preferred embodiment, convex lower surface 30 extends past concave top surface 22. In the illustrated embodiment, convex lower surface 30 and concave upper surface 22 substantially form a ball-and-socket joint. The lower surface 30 may also be in the shape of various other curves and lines including but not limited to saddle shape curves, straight lines and cylindrical curves as well as combinations of the same.

The radius of both the convex lower surface 30 and concave arch 22 are preferably relatively large. The result is a relatively large surface contact area between the two elements that protects against migration and subsidence. A shoulder 34 may extend circumferentially around convex lower surface 30 and include a lip portion 36. The shoulder 34 and lip portion 36 may limit the articulation of end plate 10 along core 20 as a result of contact between shoulder 34 and a portion of core 20 as illustrated in FIG. 2 or FIG. 4.

The resultant ball-and-socket design enables the present invention to articulate in the anterior/posterior direction, medial/lateral direction, as well as associated axial and sagittal angulations.

In a method of use, disc 1 of the present invention is disposed between two adjacent vertebrae, not shown in the drawings, with the upper surface 32 of end plate 10 abutting an upper vertebrae and a bottom surface 13 of end plate 12 abutting the lower vertebrae. Disc 1 is placed between the two vertebrae and anchored within. As shown in FIG. 2, the medial-lateral center of rotation is preferably disposed in the center of disc 1. In a preferred embodiment, the angle of articulation A′ is about 19 degrees permitting between about ±9.5 degrees from a central balanced position illustrated in FIG. 1. The disc may articulate due to movement by adjacent vertebrae against the disc.

As shown in FIG. 2, movement of an upper vertebra may cause a force F to be applied against the disc. Upon reaching the maximum articulation point, shoulder 34 and lip portion 36 preferably abut an edge of core 20 preventing any further articulation. In a preferred embodiment, as end plate 10 articulates on core 20, core 20 may slide or migrate along end plate 12 along the direction X. For example, as the medial/lateral force is applied by the vertebrae and top end plate 10 articulates on core 20, the force F may also cause core 20 to slide along end plate 12. The core may slide until protrusions 16 abut a side wall of indents 24. In a preferred embodiment, the maximum migration of core 20 relative to end plate 12 is approximately 2 mm or 1 mm in either direction. Although the present invention has been described with reference to a force created by a top vertebrae, the resultant interaction between the two end plates and core may also be caused by a lower vertebrae.

FIGS. 3 and 4 are cross-sectional side views of disc 1 showing a front view of the medial side adjacent an anterior side A and a posterior side P. In a preferred embodiment, the center of rotation C of the disc in the anterior/posterior direction is positioned slightly to the posterior as measured from a longitudinal axis 38 passing through the center of end plates 10 and 12. In a preferred embodiment, the flexion/extension range in the anterior/posterior direction is also between about ±9.5° allowing for an articulation angle A″ of about 19° as illustrated in FIG. 4. The disc may articulate in the anterior/posterior direction similarly as earlier described with reference to the medial/lateral direction. Similar to movement in the medial and lateral direction, movement in the anterior and posterior may also include sliding of core 20 relative to end plate 12 in the direction Y.

As illustrated in FIG. 4, disc 1 may articulate and slide in the anterior/posterior direction similar to the movement in the medial/lateral direction. For example, when an anterior force F′ is placed downward against end plate 10, end plate 10 articulates on core 20 while core 20 may translate on end plate 12 along the direction Y.

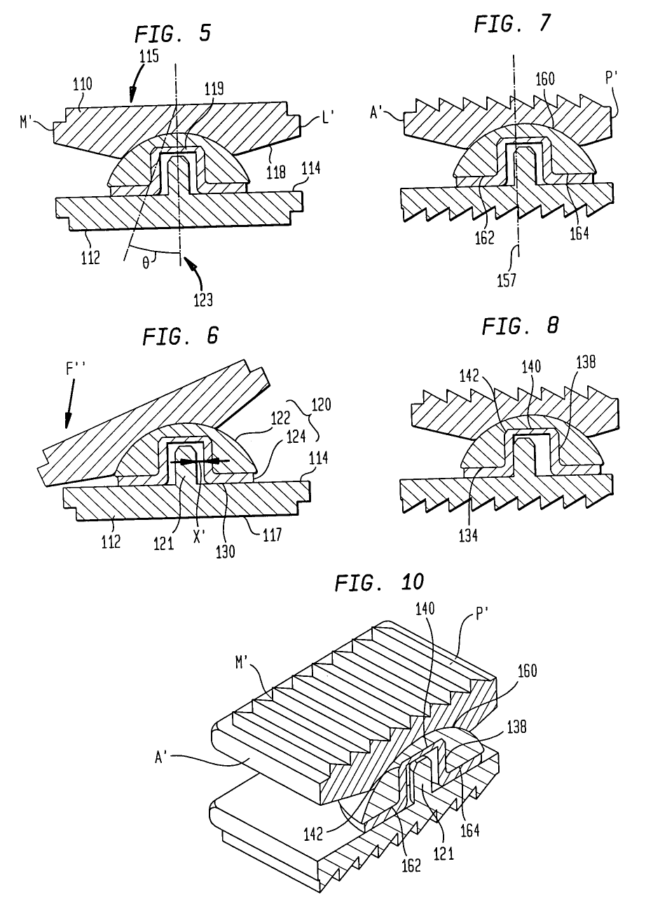

As shown in FIG. 5, disc 1 may also include a plurality of teeth 50 disposed on bottom surface 13 and upper surface 32. The teeth 50 preferably have an angled incline in the direction of the posterior to the anterior. This configuration preserves the integrity of the end plates.

Additionally the design offers a strong primary anchorage system and reduces the risk of expulsion of the disc from the adjacent vertebrae.

The present design not only enables articulation in all four directions, i.e., anterior/posterior and medial/lateral as well as axial and sagittal angulation, but also increases maneuverability of the disc by providing sliding translation in the various directions as well. Additionally, by placing a compressionable material between the two end plates, some of the upward and downward forces placed on the adjacent vertebrae may be absorbed by the compressionable disc.

The disc may also be sprayed with a coating such as a titanium plasma spray to increase bone ingrowth.

Although disc 1 has been described with reference to a core comprised of a single piece, the core may include 2 or more pieces. And the core may include two articulating surfaces, which may or may not confront each other. If the two articulating surfaces confront one another, the end plates of the present invention may both be designed with non-articulating surfaces, i.e., translation surfaces.

In an alternate embodiment, the core 20 may include more than 2 elements. For instance the core 20 may include a spherical or circular object sandwiched by two members. The two members may have arcuate surfaces confronting the spherical object and flat surfaces remote therefrom.

In an alternate embodiment, as shown in FIGS. 5-8, disc 100 may include end plates 110 and 112. End plate 110 includes top surface 115 and bottom surface 116. Bottom surface 116 preferably includes a decline ridge 118 extending downwardly toward the center of the disc and circumferentially around bottom surface 116. Bottom surface 116 preferably also includes spherical concave surface 119 adjacent to ridge 118.

As seen in FIG. 6, end plate 112 preferably includes a top surface 114 and a bottom surface 117. Top surface 114 preferably includes at least one protrusion 121 extending upwardly from top surface 114.

Disc 100 also preferably includes a core 120 disposed between end plate 110 and 112. Core 120 may be a single element or preferably consist of a bi-component having a spherical portion 122 and a base portion 124. Although the core 120 has been described including one or two elements, additional elements may also be included or comprise core 120.

In a preferred embodiment, base portion 124 is disposed on top surface 114 of end plate 112. Specifically, lower surface 130 of base portion 124 abuts top surface 114. Base portion 124 preferably includes ridge 134 extending circumferentially around the base portion at the base portion's latter edges as shown in FIG. 8. The base portion also includes shoulder 138 extending radial upwardly from ridge 134. Platform 140 is adjacent to shoulder 138 and may include a chamfered edge 142 extending radially about platform 140. Base portion 124 also preferably includes recess 150 disposed on lower surface 130 and defined by shoulder 138 and platform 140. Recess 150 is sized to be able to receive protrusion 121 of end plate 112. Preferably, recess 150 has a greater length and width compared to protrusion 121.

Spherical portion 122 is designed to cooperate with base portion 124. Spherical portion 122 preferably includes a upper convex surface 160 as shown in FIG. 7. Lower surface 162 of spherical portion 122 preferably includes floor 164 which is disposed on ridge 134 when assembled. Circumferential wall 166 extends radially upward from the inner end of floor 164. Ceiling 168 is adjacent to circumferential wall 166 and disposed on top of the same. Floor 164, wall 166 and ceiling 168 are designed to be juxtaposed against ridge 134, shoulder 138 and platform 140, respectively, so that spherical portion 122 is received by base portion 124 in a male-to-female type relationship.

In the preferred embodiment, convex surface 160 of spherical portion 122 is juxtaposed against concave surface 119 of end plate 110. The concavity of the two elements should be substantially equal much in the same way as a ball joint configuration. Additionally, as with the first embodiment, the radius of both surfaces is preferably large to create a large surface contact area.

As seen in FIG. 6, the disc of the present embodiment may have an angle of articulation θ in the medial/lateral direction. In the preferred embodiment, the center of rotation of the disc in the medial/lateral direction is along a longitudinal axis 123 passing through the center of the disc between the medial side M′ and lateral side L′ as shown in FIG. 5. In the preferred embodiment, the maximum angle of articulation θ is ±20° about the center of rotation.

In a method of use, the present embodiment of the disc is disposed between two adjacent vertebrae with the upper vertebrae contacting the top surface 115 of end plate 110 and the lower vertebrae abutting the bottom surface 117 of end plate 112. Thus, similar to the first embodiment, when a person bends from side to side, a force may be placed against the upper end plate 110 by a vertebral body as designated by F″. As the force F″ is increased, end plate 110 articulates about spherical portion 122, specifically convex surface 160. The maximum articulation angle preferably is about 20°. As end plate 110 articulates, core 120 may translate or slide on top surface 114. The core 120 may slide until protrusion 118 abuts shoulder 138 of core 120. In the preferred embodiment, this maximum translation distance is approximately 2 mm along the direction X′ or 1 mm from a center position.

Although the present embodiment has been described with reference to a medial or lateral force, the disc may also articulate and translate with regard to an anterior or posterior force as well as any force having components in a combination of directions.

FIG. 7 is a cross-sectional front view of the medial side of the disc having an adjacent anterior edge A′ and an adjacent posterior edge P′. As seen in FIG. 7, in a preferred embodiment, the center of rotation of the disc with regard to the anterior/posterior direction is slightly posterior of a longitudinal axis 157 passing through the center of end plates 110 and 112. In a preferred embodiment, the maximum articulation angle about longitudinal axis 157 is ±20°. The maximum translation distance is 2 mm along the direction Y or 1 mm from a center point.

The embodiment as illustrated in FIGS. 5-8, specifically, convex surface 119 and concave surface 160, is similar to a ball-and-joint structure. This design enables the disc to have axial rotation as well as sagittal angulation. Such movement may only be limited by the constraints of the vertebrae above and below the disc implant.

In the preferred embodiment, core 120 of disc 100 is comprised of a compressionable material such as, but not limited to, an ultra-high molecular weight polyethylene. The resultant design enables the disc to absorb some of the upward and downward force received by the vertebrae positioned adjacent disc 100.

In additional embodiments, the core, the base portion and spherical portion may consist of different materials. Preferably, at least one element is comprised of a compressionable material.

In a method of implanting disc 10, two vertebrae may be distracted using various tools known to those in the art. The distracting method may include placing trial spacers between the adjacent vertebrae until a desired distraction space is reached. The trial spacers may have the same outer contour as the disc 1 so that the correct positioning and alignment can be tested by the surgeon. The disc 1 may be inserted into the disc space by pushing the disc through two opposing rails at least partially positioned within the disc space or adjacent thereto.

Although the invention herein has been described with reference to particular embodiments, it is to be understood that these embodiments are merely illustrative of the principles and applications of the present invention. It is, therefore, to be understood that numerous modifications may be made to the illustrative embodiments and that other arrangements may be devised without departing from the spirit and scope of the present invention as defined by the appended claims.

1. An intervertebral disc comprising:
a first end plate having a first surface for engaging a vertebral body and an oppositely-facing second surface remote from said first surface, said second surface having an articulation portion, said articulation portion having a first position defining a maximum articulation angle;
a second end plate having a first surface for engaging a vertebral body and an oppositely-facing second surface remote from said first surface;
a core having an articulation surface and an oppositely-facing translation surface, said core being positioned between said first end plate and said second end plate such that said articulation portion of said second surface is positioned adjacent said articulation surface of said core for articulation therebetween, said translation surface of said core confronting said second surface of said second end plate; and
wherein said articulation portion is adapted to reach said first position in response to a force in a first direction, and said core is adapted to translate along said second surface of said second end plate in response to a force in a first direction after said articulation portion reaches said first position.

2. The intervertebral disc according to claim 1, wherein said first end plate includes a shoulder extending at least partially about said articulation portion, wherein as said force is applied in said first direction after said articulation portion has reached said first position said shoulder exerts a force against said core thereby causing said core to translate along said second end plate.

3. The intervertebral disc according to claim 1, further comprising a limiting means adapted for limiting the ability of said core to translate along said second end plate.

4. The intervertebral disc according to claim 1, wherein said articulation portion of said first end plate and said articulation surface of said core form a ball and socket joint.

5. The intervertebral disc according to claim 1, wherein said core is comprised of a polyethylene and said end plates are comprised of a metal.

6. The intervertebral disc according to claim 1, wherein said articulation surface of said core is convex.

7. The intervertebral disc according to claim 1, wherein said articulation surface of said core is concave.

8. The intervertebral disc according to claim 1, wherein said core includes a first element having a recess and a second element having a raised shoulder, said raised shoulder being disposed in said recess of said first element.

9. An intervertebral disc comprising:
a first end plate having a first surface for engaging a vertebral body and an oppositely-facing second surface remote from said first surface, said second surface having an articulation portion;
a second end plate having a first surface for engaging a vertebral body and an oppositely-facing second surface remote from said first surface, said second surface having at least one protrusion extending outwardly therefrom, said protrusion having a height, a width and a length, each having a first dimension; and
a core having an articulation surface and an oppositely-facing translation surface, said translation surface including at least one indent having a height, a length and a width, each having a second dimension, said height dimension of said indent being greater than said height dimension of said protrusion and at least one of said length dimension or said width dimension of said protrusion being greater than said height dimension or said width dimension of said protrusion respectively, said core being positioned between said first end plate and said second end plate such that said articulation surface of said core confronts said second surface of said first end plate and said translation surface of said core confronts said second surface of said second end plate, said core capable of articulating relative to said first end plate and translating relative to said second end plate, said at least one protrusion of said second surface being disposed within said at least one indent of said translation surface, wherein said protrusion limits translation of said core across said second end plate.

10. The intervertebral disc according to claim 9, wherein said length dimension and said width dimension of said indent are both greater than said length dimension and said width dimension of said protrusion, respectively.

11. The intervertebral disc according to claim 9, wherein said core has at least two indents and said second end plate has two protrusions that are disposed within said at least two indents.

12. The intervertebral disc according to claim 9, wherein said articulation portion of said first end plate and said articulation surface of said core form a ball and socket.

13. The intervertebral disc according to claim 9, wherein said articulation portion of said first end plate is convex.

14. The intervertebral disc according to claim 13, wherein said first end plate includes a shoulder extending at least partially around said articulation portion, wherein said articulation portion has a first position directed to a maximum articulation angle, wherein as said articulation portion approaches said first position, at least a portion of said shoulder approaches said core.

15. An intervertebral disc comprising:
a first end plate having a vertebral body contacting surface and an articulation surface spaced therefrom;
a core element having an articulation surface for articulation with said articulation surface of said first end plate, said core element having a translation surface spaced from said articulation surface, said translation surface having a stop element formed therein; and
a second end plate having a vertebral body contacting surface and a translation surface spaced therefrom, said translation surface slidably engaging said core translation surface, said end plate translation surface including a stop element engagable with said core stop element to limit the relative translation between said core element and said second end plate.

16. The intervertebral disc as set forth in claim 15, wherein said articulation surface of said first end plate includes a stop element and said articulation surface of said core element includes a stop element engagable with said first end plate stop element to limit the articulation therebetween.

17. The intervertebral disc as set forth in claim 16, wherein the articulation surface of said core and said first end plate have a slope being part of a surface of revolution.

18. The intervertebral disc as set forth in claim 15, wherein said translation stop elements are in the form of a circular projection and a circular recess formed, respectively, on one of said core or said second end plate.

19. The intervertebral disc as set forth in claim 18, wherein said articulation stop elements are in the form of a circumferential edge formed on said core and a circumferential groove formed on said first end plate.

20. An intervertebral disc comprising:
a first portion having a first surface for engaging a vertebral body and an oppositely-facing second surface remote from said first surface,
a second portion having a first surface for engaging a vertebral body and an oppositely-facing second surface remote from said first surface, said first surface having a stop element disposed thereon; and
a core element having an articulation surface and a translation surface, said translation surface having a stop element engagable with said stop element of said second portion, said translation surface slidably engaging said first surface of said second portion as said core stop element engages said second portion stop element.
